SEQUENCE LISTING (12 sequences)

SEQ ID NO: 1 - DNA, 486 bp, Lotus japonicus
  CDS (1)..(486)
atgtccacccttgggagcacatgtttcactgaagagcaagaagctcttgtggtcaagtcatggagcgtgatgaagaagaattctgctgaactgggtctcaagcttttcttgaaaatatttgagattgctccatcagctcagaaattgttctctttcttgagagattcaaaagttcctttggaggagaaccccaagctcaagcctcatgccatgtctgtctttgtcatgacttgtgaatcagcagctcaactgcggaaggctggaaaagtcactgtgagagaatcaaccttgaaaaagctaggtgctacccattataaatatggagtagtaaacgagcattttgaggttacaaagtttgcactactggataccataaaagaagctgtacctgaaatgtggtcaccagagatgaagaatgcatggacacaagcttatgatcagctggtcggtgccattaaatctgaaatgaagccatcatcttcttag

SEQ ID NO: 2 - protein, 161 aa, Lotus japonicus
MSTLGSTCFTEEQEALVVKSWSVMKKNSAELGLKLFLKIFEIAPSAQKLFSFLRDSKVPLEENPKLKPHAMSVFVMTCESAAQLRKAGKVTVRESTLKKLGATHYKYGVVNEHFEVTKFALLDTIKEAVPEMWSPEMKNAWTQAYDQLVGAIKSEMKPSSS

SEQ ID NO: 3 - DNA, 1012 bp, Lotus japonicus
atgtccacccttgggagcacatgtttcactgaagagcaagaagctcttgtggtcaagtcatggagcgtgatgaagaagaattctgctgaactgggtctcaagcttttcttgaagtaagtcatttaatttcatcaacattataagttcatggcatatatagttgtattttcatttttgtccttagacaacaatgtgctatttagaacaattgaattgataataaaaattgtgtctttgtagaatatttgagattgctccatcagctcagaaattgttctctttcttgagagattcaaaagttcctttggaggagaaccccaagctcaagcctcatgccatgtctgtctttgtcatggtaaccttacttcctttcactctgtctccattcaccatttgaatgaatgataaatactatgtttagattaactactcttttctctttcatcagaatctaccaaggagcttctcatgattttggttacaaaataaattgtagaagaattttcgaatacgaaatcagataaattaagaagctttattttgtttttgttattgcagacttgtgaatcagcagctcaactgcggaaggctggaaaagtcactgtgagagaatcaaccttgaaaaagctaggtgctacccattataaatatggagtagtaaacgagcattttgaggtatatatataaaatttcaattagtatgtttgagaaagcattgtttatttgaaagtgtcaacatatacaagattgaaaatgacatttgctatattaacttttacatcaagttgtataaaacaatgtaatcttgcatatggagtgctttttgtatttgatggggcatatgtataatttattctttggttttgtaaaggttacaaagtttgcactactggataccataaaagaagctgtacctgaaatgtggtcaccagagatgaagaatgcatggacacaagcttatgatcagctggtcggtgccattaaatctgaaatgaagccatcatcttcttag

SEQ ID NO: 4 - DNA, 21 bp, Artificial Sequence
  Synthetic primer
ttctcacttcacttccatcgc

SEQ ID NO: 5 - DNA, 19 bp, Artificial Sequence
  Synthetic primer
ttggtcaagtcatggagcg

SEQ ID NO: 6 - DNA, 20 bp, Artificial Sequence
  Synthetic primer
tcacagtgacttttccagcg

SEQ ID NO: 7 - DNA, 20 bp, Artificial Sequence
  Synthetic primer
agacagacatggcatgaggc

SEQ ID NO: 8 - DNA, 483 bp, Alnus firma
  CDS (1)..(483)
atgaacaccttggaaggaagagggttcacagaagagcaagaagctgtggtggtgaagtcatggaatgcaatgaagcctaatgctggagaattgggtctaaaattcttcttgaagatatttgagattgcaccatcagctcagaagctcttctctttcttgagagactcaaatgtttctctcgaacgaaatccaaagcttaagtcccatgccatgtctgtctttctgatgacctgtgaatcggcagtgcaactccggaaagccggcaaagttactgtgagagagtcgagcttgaaaaagttgggtgctgtccacttcaagcatggggtggtcgatgaacattatgaggtcacaaagtttgcgctgctggaaactatcaaggaagcggtgccagaaatgtggtcaccggagatgaagattgcatggggagaagcttatgatcagttggttgctgctatcaaatctgcaatgaagccttcttcttag

SEQ ID NO: 9 - protein, 160 aa, Alnus firma
MNTLEGRGFTEEQEAVVVKSWNAMKPNAGELGLKFFLKIFEIAPSAQKLFSFLRDSNVSLERNPKLKSHAMSVFLMTCESAVQLRKAGKVTVRESSLKKLGAVHFKHGVVDEHYEVTKFALLETIKEAVPEMWSPEMKIAWGEAYDQLVAAIKSAMKPSS

SEQ ID NO: 10 - DNA, 892 bp, Alnus firma
atgaacaccttggaaggaagagggttcacagaagagcaagaagctgtggtggtgaagtcatggaatgcaatgaagcctaatgctggagaattgggtctaaaattcttcttgaagtaagccatattaataaatttatcgtttgattataattaatttcccatatatatcaatggctgttgatttgaacaaatgggttttgtaggatatttgagattgcaccatcagctcagaagctcttctctttcttgagagactcaaatgtttctctcgaacgaaatccaaagcttaagtcccatgccatgtctgtctttctgatggtaagacccctttgagtgatttgaatcgagattatctatgttgtagtttcatggtttttggtttgtttgaaagatatggtttcatggtttggtaggctaagacggctatgaaatctgtgagagttaatcttctaattgattggccggaaagtggaaacctattaattaagaataagcatgtaatcaattgtgtttggttaattttgtatatgtatattgcagacctgtgaatcggcagtgcaactccggaaagccggcaaagttactgtgagagagtcgagcttgaaaaagttgggtgctgtccacttcaagcatggggtggtcgatgaacattatgaggtacgacgtagtatcctcatccatccatgcatttgctcatatatccctgctgattaattatttccatggctgatcatggctttgtaaatggttcacaggtcacaaagtttgcgctgctggaaactatcaaggaagcggtgccagaaatgtggtcaccggagatgaagattgcatggggagaagcttatgatcagttggttgctgctatcaaatctgcaatgaagccttcttcttag

SEQ ID NO: 11 - DNA, 20 bp, Artificial Sequence
  Synthetic primer
gctgctatcaaatctgcaat

SEQ ID NO: 12 - DNA, 18 bp, Artificial Sequence
  Synthetic primer
ggggggctgtgattttag

(19) United States
(12) Patent Application Publication
(10) Pub. No.: US 2008/0295198 A1
(43) Pub. Date: Nov. 27, 2008

(54) Method for Producing Plant Forming Nodules with High Nitrogen-Fixing Activity

(76) Inventors: Toshiki Uchiumi, Kagoshima (JP); Yoshikazu Shimoda, Kagoshima (JP)

Correspondence Address: BIRCH STEWART KOLASCH & BIRCH, PO BOX 747, FALLS CHURCH, VA, 22040-0747, US

(21) Appl. No.: 11/886,249

(22) PCT Filed: Mar. 3, 2006
(86) PCT No.: PCT/JP06/04681

§ 371 Date: Sep. 13, 2007

(30) Foreign Application Priority Data
Mar. 14, 2005 (JP) 2005-071677

(51) Int. Cl. A01H 1/06; A01H 5/00; C12N 15/63

(52) U.S. Cl. 800/278; 800/295; 435/320.1

(57) ABSTRACT

The present invention relates to a method for producing a nodulating plant capable of forming nodules with enhanced nitrogen-fixing activity, comprising causing overexpression of a nonsymbiotic globin gene in a nodulating plant.

### TECHNICAL FIELD

The present invention relates to a method for producing a nodulating plant that forms nodules with high nitrogen-fixing activity.

### BACKGROUND ART

Leguminous crops such as Glycine max (soybean), Phaseolus angularis (azuki bean), and Phaseolus vulgaris (kidney bean) form nodules (nodulation), which are symbiotic organs, through infection with root nodule bacteria. Bacteroids of symbiotic root nodule bacteria within such nodules fix atmospheric nitrogen, so that the plants can grow well even in soil with low nitrogen content. Hence, when a leguminous crop is cultivated, the seeds of the crop are often treated with pre-cultured root nodule bacteria in addition to an application of a general fertilizer. To further enhance the nitrogen-fixing ability of the nodules of leguminous crops, root nodule bacteria with enhanced nitrogen-fixing ability have also been developed (JP Patent Publication (Kokai) No. 2003-33174 A).

In addition to leguminous crops, it is known that symbiotic nitrogen-fixing bacteria can invade and proliferate in the roots of leguminous trees such as the genus Acacia and Albizia julibrissin and nonleguminous trees such as Alnus japonica and Alnus firma, form nodules, efficiently fix atmospheric nitrogen, and thus supply nitrogen to the host trees. These nodulating trees have high nitrogen concentrations in their living leaves and thus have high nitrogen concentrations in fallen leaves. Accordingly, nodulating trees are effective in increasing microbial levels in soil and improving the quality of the soil so as to fertile the soil. Thus, such trees are also used as the so-called soil-improving trees for greening waste land.

Enhancement of the nitrogen-fixing ability of nodules of such plants is thought to be very useful in terms not only of agriculture but also of environmental conservation.

Meanwhile, it is known that in the nodule cells of leguminous plants, symbiotic globin genes that are unique in leguminous plants are very strongly expressed. Symbiotic hemoglobin (also referred to as leghemoglobin) is composed of a globin that is a gene product of such a symbiotic globin gene and heme, and it is said to account for 20% to 30% of all the soluble proteins in the nodules. But the symbiotic hemoglobin is completely absent in tissues other than the nodules. Symbiotic hemoglobin has strong affinity for oxygen and the like, as with hemoglobin in animal blood. The symbiotic hemoglobin is believed to serve a function of regulating the oxygen partial pressure within nodule cells at a level that is sufficient for respiration of root nodule bacteria within nodules and that prevents nitrogenase from being inactivated since the nitrogenase is required for the nitrogen-fixing ability of root nodule bacteria, but can be inactivated by oxygen.

With the recent development of gene analysis technology, it has been revealed that plants other than leguminous plants also have globin genes. Such globin genes discovered in plants other than leguminous plants differ from the symbiotic globin genes of leguminous plants. Thus such globin genes have been termed “nonsymbiotic globin genes” (also referred to as nonsymbiotic hemoglobin genes). It is currently believed that all plants have nonsymbiotic globin genes. In other words, leguminous plants have both symbiotic globin genes and nonsymbiotic globin genes, but non-leguminous plants have nonsymbiotic globin genes alone. A nonsymbiotic globin gene of Lotus japonicus that is a model plant belonging to the family Leguminoseae has also been reported (Uchiumi et al., Plant Cell Physiol. (2002) 43(11): pp. 1351-1358).

Unlike symbiotic globin genes, nonsymbiotic globin genes are known to be expressed in all plant tissues. It has been reported that when a plant is exposed to a low temperature (4° C.) or low-oxygen partial pressure (at oxygen concentration of 5% or less), the expression level of its nonsymbiotic globin gene is increased. It has also been reported that resistance against low oxygen stress has been enhanced in Arabidopsis thaliana into which a nonsymbiotic globin gene has been introduced and overexpressed (Hunt, P. W., et al., Proc. Natl. Acad. Sci. (2002) U.S.A. 99: pp. 17197-17202).

### DISCLOSURE OF THE INVENTION

An object of the present invention is to provide a method for producing a nodulating plant that forms nodules having enhanced nitrogen-fixing activity, and a nodulating plant that exhibits high nitrogen-fixing activity of its nodules.

As a result of intensive studies to achieve the above object, the present inventors have discovered that Lotus japonicus into which a Lotus japonicus nonsymbiotic globin gene has been introduced and overexpressed, and Lotus japonicus into which an Alnus firma nonsymbiotic globin gene has been introduced and overexpressed can form nodules having high nitrogen-fixing activity. The present inventors have completed the present invention based on such findings.

The present invention encompasses the following.

[1] A method for producing a nodulating plant capable of forming nodules having enhanced nitrogen-fixing activity, comprising causing overexpression of a nonsymbiotic globin gene in a nodulating plant.

More preferably, the nonsymbiotic globin gene in this method comprises a DNA selected from the group consisting of the following (a) to (e):

(a) a DNA comprising the nucleotide sequence shown in SEQ ID NO: 1 or 8;

(b) a DNA which hybridizes under stringent conditions to a DNA comprising a nucleotide sequence complementary to the nucleotide sequence shown in SEQ ID NO: 1 or 8 and encodes a protein having nonsymbiotic globin activity;

(c) a DNA encoding a protein comprising the amino acid sequence shown in SEQ ID NO: 2 or 9;

(d) a DNA encoding a protein that comprises an amino acid sequence derived from the amino acid sequence shown in SEQ ID NO: 2 or 9 by deletion, substitution, or addition of 1 to 50 amino acids and has nonsymbiotic globin activity; and

(e) a DNA comprising the nucleotide sequence shown in SEQ ID NO: 3 or 10.

In this method, preferably, the overexpression of the nonsymbiotic globin gene is caused by introducing the nonsymbiotic globin gene ligated to an overexpression promoter into a nodulating plant.

According to this method, preferably, a nodulating plant capable of forming nodules having an enhanced nitrogen-fixing activity at least 3 times greater than that of a wild-type strain can be produced. Such nodulating plant that is produced according to this method can form nodules having enhanced nitrogen-fixing activity, preferably nodules that exhibit nitrogen-fixing activity of 10 nM/min/g to 100 nM/min/g.

The nodulating plant in which the overexpression of the nonsymbiotic globin gene is caused in this method is preferably a leguminous plant. More preferably, according to this method, the nodulating plant which has overexpressed the nonsymbiotic globin gene is further inoculated with a symbiotic nitrogen-fixing bacterium.

[2] A nodulating plant which is produced according to the method of [1] above, wherein the nodulating plant is capable of forming nodules having enhanced nitrogen-fixing activity. Such nodulating plant more preferably has nodules on its roots.
[3] A vector for enhancing the nitrogen-fixing activity of nodules, comprising a nonsymbiotic globin gene ligated to an overexpression promoter.

The nonsymbiotic globin gene to be contained in the vector is preferably derived from a leguminous plant or a nonleguminous nodulating plant. The nonsymbiotic globin gene comprises more preferably the DNA described in (a) to (e) of [1] above.

[4] A method for enhancing nitrogen-fixing efficiency upon plant cultivation, comprising cultivating the nodulating plant of [2] above.

According to the method for producing a nodulating plant of the present invention, the nitrogen-fixing activity of nodules of a nodulating plant of interest can be markedly improved. The vector of the present invention for enhancing the nitrogen-fixing activity of nodules can, when it is used in such production method, contribute to significant enhancement of the nitrogen-fixing activity of the nodules. Furthermore, a nodulating plant obtainable according to the present invention is capable of forming nodules having high nitrogen-fixing activity, resulting in promoted growth of the plant and increased nitrogen content. The present invention also relates to a method for increasing the nitrogen fixation level upon plant cultivation through cultivation of the nodulating plant, and the method makes it possible not only to increase the amount of nitrogen to be fixed from atmosphere in a given environment and to increase the yield or the growth amount of the relevant nodulating plant under such environment, but also to increase the amount of nitrogen in soil and thus fertilize the soil.

This description includes the disclosure of the description and drawings of Japanese Patent Application No. 2005-071677, from which the present application claims priority.

### BRIEF DESCRIPTION OF THE DRAWINGS

FIG. 1 shows the genomic structure and the amino acid sequence of Lotus japonicus nonsymbiotic globin gene.

FIG. 2 shows expression levels of a nonsymbiotic globin gene in wild-type Lotus japonicus. FIG. 2A shows the expression levels indifferent tissues. FIG. 2B shows the expression levels under stress conditions.

FIG. 3 shows the structure of a transformation vector used for transformation of hairy roots.

FIG. 4 shows photographs showing experimental results of confirming the introduction of nonsymbiotic globin gene in transformants. FIG. 4A shows hairy root induction in transformants. FIG. 4B shows GFP fluorescence as observed in transformant roots. FIG. 4C shows the expression of the transgenes as confirmed by RT-PCR.

FIG. 5 shows photographs showing nodules formed on transformed hairy roots. FIG. 5A shows Lotus japonicus hairy roots (control roots) obtained as a result of introduction of a vector not containing an LjHb1 gene. FIG. 5B shows hairy roots into which the LjHb1 gene has been introduced and overexpressed. White arrowhead marks indicate nodules formed on transformed hairy roots. Mesh arrowhead marks indicate nodules formed on non-transformed hairy roots. It is shown that only the transformed nodules emitted fluorescence.

FIG. 6 shows ethylene amount data measured by gas chromatography, showing the nitrogen-fixing activity (ARA activity) levels of nodules formed on transformed hairy roots.

FIG. 7 is a schematic diagram showing a vector used for expression of Lotus japonicus globin proteins in Escherichia coli.

FIG. 8 shows absorbance spectra showing affinity of Lotus japonicus globin proteins (symbiotic [left graph, FIG. 8A] and nonsymbiotic [right graph, FIG. 8B]) expressed in Escherichia coli, for nitrogen monoxide. Each line denotes data obtained via measurement at different times for mixing of the globin proteins with nitrogen monoxide.

FIG. 9 shows photographs showing the expression of AfHb1 transgene in Lotus japonicus transformants as confirmed by RT-PCR.

FIG. 10 shows nitrogen-fixing activity (ARA activity) levels in whole transformed plants (Lotus japonicus) into which AfHb1 has been introduced and the same in the thus formed nodules.

### BEST MODE FOR CARRYING OUT THE INVENTION

Hereinafter, the present invention will be described in detail.

### 1. NODULATING PLANT

According to the present invention, a nodulating plant capable of forming nodules having enhanced nitrogen-fixing activity is produced by causing overexpression of a nonsymbiotic globin gene in a nodulating plant. In the Description, “nodulating plant” means a plant of a species that is capable of nodulating or forming nodules. Nodules are symbiotic organs with granular structures formed due to the inhabitation of symbiotic nitrogen-fixing bacteria in the roots of nodulating plants. Examples of such nodulating plants include not only leguminous plants (including leguminous crops and leguminous trees), but also some nonleguminous plants (mainly nonleguminous trees) such as members of the family Betulaceae or the family Alnus, which are generically named actinorhizal plants. Root nodule bacteria live symbiotically in the roots of a leguminous plant as symbiotic nitrogen-fixing bacteria, forming nodules. In the case of some nonleguminous plants mentioned above, Actinomycetes live symbiotically as symbiotic nitrogen-fixing bacteria in the roots, forming nodules. Examples of nodulating plants useful in the present invention include, when they are leguminous plants, Lotus japonicus, Glycine max (soybean), Phaseolus angularis (azuki bean), Phaseolus vulgaris (kidney bean), Pisum sativum (pea), Vicia faba, Arachis hypogaea, Medicago sativa (alfalfa), Medicago truncatula, the genus Trifolium (clover), Vigna sinensis, Lens esculenta (lentil), Robina pseudoacacia, Cytisus scoparius, the genus Lespedeza, Sophora japonica, and Paraserianthes Falcataria. Examples of nodulating plants useful in the present invention include, when they are non-leguminous plants, Alnus firma, Alnus japonica, Myrica rubra, Casuarina stricta, Coriaria japonica, and the genus Elaeagnus.

In the present invention, nodulating plants may be either plant bodies with nodules or plant bodies with no nodules at a time point at which a nonsymbiotic globin gene is introduced, at which the plant body is regenerated, or any other time points, as long as they are plants of biological species capable of nodulating or forming nodules.

In the Description, the term “nodulating plant” refers not only to nodulating plant bodies (whole plants), but also plant organs (e.g., leaves, petals, stems, roots, seeds, hypocotyls, and cotyledons), plant tissues (e.g., epidermis, phloem, parenchyma, xylem, vascular bundles, palisade tissues, and spongy tissues), and any portions of plant bodies such as cultured plant cells (e.g., calli), for example.

### 2. NONSYMBIOTIC GLOBIN GENE AND OBTAINMENT THEREOF

The nonsymbiotic globin gene according to the present invention encodes a globin protein that becomes associated with heme (a complex salt of porphyrin and ferrous ion) to form nonsymbiotic hemoglobin. As with animal hemoglobin, nonsymbiotic hemoglobin is a protein having strong affinity for oxygen, carbon dioxide, carbon monoxide, or the like. The nonsymbiotic hemoglobin has stronger affinity for oxygen, nitrogen monoxide, or the like than symbiotic hemoglobin. In the Description, the activity of globin encoded by such nonsymbiotic globin gene is referred to as nonsymbiotic globin activity.

The nonsymbiotic globin gene according to the present invention can be isolated from any plant. The nonsymbiotic globin gene of the present invention is more preferably derived from a nodulating plant. The nonsymbiotic globin gene may be isolated from a leguminous plant such as Lotus japonicus or Glycine max (soybean) or may be isolated from a nonleguminous nodulating plant such as Alnus firma. Such nonsymbiotic globin gene to be used in the present invention may also be isolated from a plant other than a nodulating plant, such as a monocotyledon (e.g., Hordeum vulgare (barley), Oryza sativa (rice), or Zea mays (corn)). Examples of a known nonsymbiotic globin gene that can be used in the present invention are as follows: Lotus japonicus (Uchiumi et al., Plant Cell Physiol. (2002) 43(11): pp. 1351-1358), Alnus firma (DDBJ/EMBL/GenBank accession No. AB221344), Oryza sativa (rice) (DDBJ/EMBL/GenBank accession No. U76030), Medicago sativa (alfalfa) (DDBJ/EMBL/GenBank accession No. AF172172; Serogelyes et al., FEBS Lett. (2000) 482, pp. 125-130), Glycine max (soybean) (DDBJ/EMBL/GenBank accession No. U47143; Anderson, et al., Proc. Natl. Acad. Sci. U.S.A. (1996) 93(12), pp. 5682-5687), and Arabidopsis thaliana (DDBJ/EMBL/GenBank accession No. U94998; Trevaskis et al., PNAS (1997) 94 pp. 12230-12234).

The nonsymbiotic globin gene to be used in the present invention may be a cDNA or a genomic DNA containing exons and introns. Examples of a “gene” used herein include a DNA and an RNA. Examples of such DNA include at least a genomic DNA, a cDNA, and a synthetic DNA. Examples of such RNA include an mRNA and the like. The “gene” used herein may also contain an untranslated region (UTR) sequence in addition to a coding sequence.

In a non-limiting preferred embodiment, a Lotus japonicus nonsymbiotic globin gene can be used as the nonsymbiotic globin gene derived from a leguminous plant according to the present invention. For example, a DNA comprising the nucleotide sequence of SEQ ID NO: 1 isolated from Lotus japonicus or a DNA of a genomic fragment comprising the nucleotide sequence of SEQ ID NO: 3 isolated from Lotus japonicus can be appropriately used as the nonsymbiotic globin gene of the present invention. Furthermore, a DNA encoding a Lotus japonicus nonsymbiotic globin protein that comprises the amino acid sequence of SEQ ID NO: 2 can also be advantageously used.

As the nonsymbiotic globin gene of the present invention, a DNA encoding a protein that comprises an amino acid sequence derived from the amino acid sequence shown in SEQ ID NO: 2 by deletion, substitution, or addition of 1 to 50, preferably 1 to 35, more preferably 1 or several (e.g., 2 to 10) amino acids may also be used, as long as it has nonsymbiotic globin activity. Furthermore, such nonsymbiotic globin gene to be used in the present invention may also be a DNA that hybridizes under stringent conditions to a DNA comprising a nucleotide sequence complementary to the nucleotide sequence shown in SEQ ID NO: 1 or to a DNA comprising a nucleotide sequence complementary to the nucleotide sequence of a DNA encoding the nonsymbiotic globin protein comprising the amino acid sequence of SEQ ID NO: 2 and that encodes a protein that has nonsymbiotic globin activity. The nonsymbiotic globin gene of the present invention may also be a DNA encoding a protein that comprises an amino acid sequence having at least 80% identity with the amino acid sequence of SEQ ID NO: 2 and that has nonsymbiotic globin activity.

In another embodiment, an Alnus firma nonsymbiotic globin gene can be appropriately used as the nonsymbiotic globin gene derived from a nonleguminous nodulating plant according to the present invention, but examples are not limited thereto. For example, as the nonsymbiotic globin gene of the present invention, a DNA comprising the nucleotide sequence of SEQ ID NO: 8 isolated from Alnus firma or a DNA of a genomic fragment comprising the nucleotide sequence of SEQ ID NO: 10 isolated from Alnus firma can be appropriately used. Moreover, a DNA encoding an Alnus firma nonsymbiotic globin protein comprising the amino acid sequence of SEQ ID NO: 9 can also be advantageously used.

As the nonsymbiotic globin gene of the present invention, a DNA encoding a protein that comprises an amino acid sequence derived from the amino acid sequence shown in SEQ ID NO: 9 by deletion, substitution, or addition of 1 to 50, preferably 1 to 35, and more preferably 1 or several (e.g., 2 to 10) amino acids may also be used, as long as it has nonsymbiotic globin activity. Such nonsymbiotic globin gene to be used in the present invention may be a DNA that hybridizes under stringent conditions to a DNA comprising a nucleotide sequence complementary to the nucleotide sequence shown in SEQ ID NO: 8 or to a DNA comprising a nucleotide sequence complementary to the nucleotide sequence of a DNA encoding the nonsymbiotic globin protein comprising the amino acid sequence of SEQ ID NO: 9 and that encodes a protein that has nonsymbiotic globin activity. The nonsymbiotic globin gene of the present invention may also be a DNA encoding a protein that comprises an amino acid sequence having at least 80% identity with the amino acid sequence of SEQ ID NO: 9 and that has nonsymbiotic globin activity.

In the present invention, the term “stringent conditions” refers to conditions under which namely a specific hybrid is formed. Under examples of such conditions, nucleic acids sharing high homology, and specifically, DNAs having 90% or more and preferably 95% or more homology, hybridize to each other, but nucleic acids sharing homology lower than such homology do not hybridize to each other. More specifically, such stringent conditions refer to conditions in which the sodium salt concentration ranges from 15 mM to 750 mM, preferably 50 mM to 750 mM, and more preferably 300 mM to 750 mM, the temperature ranges from 25° C. to 70° C., and preferably 50° C. to 70° C., and more preferably 55° C. to 65° C., and the formamide concentration ranges from 0% to 50%, preferably 20% to 50%, and more preferably 35% to 45%. Furthermore, under stringent conditions, post-hybridization filter-washing conditions generally are conditions in which the sodium salt concentration ranges from 15 mM to 600 mM, preferably 50 mM to 600 mM, and more preferably 300 mM to 600 mM, and the temperature ranges from 50° C. to 70° C., preferably 55° C. to 70° C., and more preferably 60° C. to 65° C.

In a non-limiting embodiment of the present invention, the nonsymbiotic globin activity of a globin protein encoded by a nonsymbiotic globin gene is represented by the affinity of the nonsymbiotic hemoglobin comprising such globin protein for oxygen, carbon dioxide, nitrogen monoxide, or the like. The nonsymbiotic globin activity of the present invention is determined as described below, for example. A globin protein is recombinantly produced using an expression vector containing the nonsymbiotic globin gene encoding the relevant nonsymbiotic globin and then nonsymbiotic hemoglobin is reconstructed using the thus obtained globin protein and heme. The affinity of the nonsymbiotic hemoglobin for oxygen, carbon dioxide, nitrogen monoxide, or the like is measured by a conventional technique and thus the measured value can be used as an indicator of the nonsymbiotic globin activity. The affinity of the nonsymbiotic hemoglobin for oxygen, carbon dioxide, or nitrogen monoxide can be measured by methods known by persons skilled in the art.

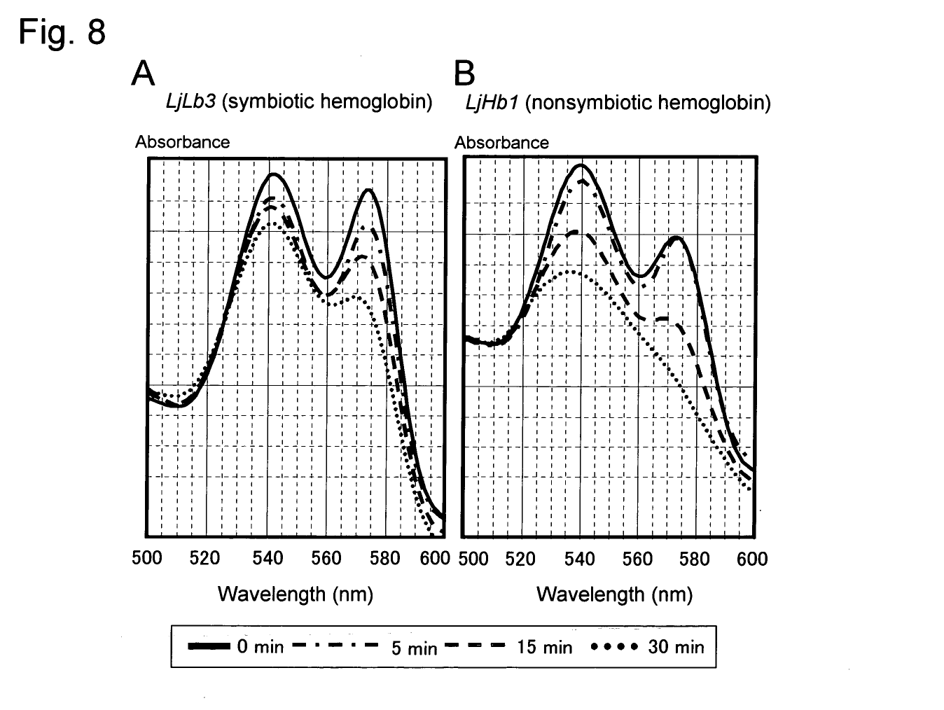

In an example, the affinity of such nonsymbiotic hemoglobin for nitrogen monoxide can be determined based on the absorbance spectrum obtained by measuring absorbance at a wavelength between 500 nm and 600 nm in the presence of nitrogen monoxide. In general, when an absorbance curve is plotted for hemoglobin using light with a wavelength between 500 nm and 600 nm, two characteristic peaks are observed at 540 nm and 575 nm (see FIG. 8). These 2 peaks disappear in the case of hemoglobin observed after the completion of the reaction with nitrogen monoxide. Hence, the affinity of a hemoglobin protein for nitrogen monoxide can be measured by mixing the hemoglobin protein with nitrogen monoxide and then analyzing the absorbance spectrum according to a conventional technique. Generally, isolated and/or purified hemoglobin is present in a form that is bound with oxygen (referred to as oxyhemoglobin). When recombinantly produced for example, nonsymbiotic hemoglobin is also isolated in a form that is bound with oxygen. The nonsymbiotic hemoglobin to be used herein has higher affinity for nitrogen monoxide than for oxygen. Therefore, when nitrogen monoxide is added to isolated nonsymbiotic hemoglobin, two peaks as observed in the absorbance spectrum disappear and then a shift to the spectrum obtained after completion of the reaction with nitrogen monoxide is observed. Regarding such method for measuring the affinity of hemoglobin for nitrogen monoxide, Michele Perazzoli et al., The Plant Cell (2004) 16, pp. 2785-2794 can be referred to.

Through comparison analysis of the absorbance spectrum measured as described above between nonsymbiotic hemoglobin and symbiotic hemoglobin, their affinities for nitrogen monoxide can be relatively compared. Comparison of the absorbance spectra reveals that nonsymbiotic hemoglobin can bind to nitrogen monoxide more rapidly than symbiotic hemoglobin. In the present invention, the affinity of nonsymbiotic hemoglobin for nitrogen monoxide can be determined using as an indicator the time required for mixing with nitrogen monoxide until the binding of nonsymbiotic hemoglobin with nitrogen monoxide is observed in the absorbance spectrum.

In the present invention, nonsymbiotic globin may be recombinantly produced in Escherichia coli using a vector containing a gene encoding such nonsymbiotic globin (SEQ ID NO: 2 or 9), and then nonsymbiotic hemoglobin that is reconstructed with the nonsymbiotic globin and heme in the Escherichia coli may be collected. The affinity of the nonsymbiotic hemoglobin for nitrogen monoxide can then be measured by the above-described method, for example. Preferably, the nonsymbiotic globin of the present invention has the same degree of affinity for nitrogen monoxide as that of nonsymbiotic hemoglobin comprising such nonsymbiotic globin (SEQ ID NO: 2 or 9), but the examples are not limited thereto.

The nonsymbiotic globin gene according to the present invention as described above can be obtained as a nucleic acid fragment by performing PCR amplification using primers designed based on the sequences of SEQ ID NOs: 1 to 3 and 8 to 10, for example, and a nucleic acid derived from any plant (e.g., a nodulating plant such as Lotus japonicus or Alnus firma) as a template. Furthermore, the nonsymbiotic globin gene of the present invention can be obtained as a nucleic acid fragment by performing hybridization using a nucleic acid derived from any plant (e.g., a nodulating plant such as Lotus japonicus or Alnus firma) as a template and a DNA fragment that is a portion of the nonsymbiotic globin gene as a probe. A nucleic acid to be used as a template in these methods may be a genomic DNA extracted from any plant by a conventional technique or a cDNA synthesized via reverse transcription from a mRNA extracted by a conventional technique, for example. A nucleic acid to be used as a template may be a purified genomic DNA, a purified cDNA, a cDNA library, a genomic DNA library, or the like. Alternatively, such nonsymbiotic globin gene to be used in the present invention may also be synthesized as a nucleic acid fragment by various nucleic acid sequence synthesis methods known in the art, such as a chemical synthesis method.

Furthermore, desired mutation may be introduced into the nucleotide sequence (ultimately, into the amino acid sequence encoded by the nucleotide sequence) of the thus obtained nonsymbiotic globin gene by site-specific mutagenesis or the like. For introduction of a mutation into a gene, known techniques such as the Kunkel method, the gapped duplex method, or any techniques based on those methods can be employed. For example, mutation may be introduced using a mutagenesis kit (e.g., Mutan-K (TAKARA) or Mutan-G (TAKARA)) utilizing site-specific mutagenesis or the LA PCR in vitro Mutagenesis Series Kit (TAKARA).

In addition, experimental protocols employed in the present invention, such as mRNA preparation, cDNA preparation, PCR, RT-PCR, library construction, ligation into vectors, transformation of cells, DNA sequencing, chemical synthesis of nucleic acids, determination of the amino acid sequence on the N-terminal side of a protein, mutagenesis, and protein extraction can be performed according to methods as described in ordinary laboratory manuals. An example of such a laboratory manual is Sambrook et al., Molecular Cloning, A Laboratory Manual, 2001, Eds., Sambrook, J. & Russell, D. W., Cold Spring Harbor Laboratory Press.

### 3. OVEREXPRESSION OF NONSYMBIOTIC GLOBIN GENES IN NODULATING PLANTS

According to the present invention, overexpression of a nonsymbiotic globin gene is caused in a nodulating plant by genetic engineering techniques. The wording “causing overexpression” used herein means that: genetically engineering a gene in a host organism by any of genetic engineering techniques (e.g., gene transfer) such that the gene is expressed at a level above the normal expression level of the gene in the host organism (e.g., higher by 10% or more); or introducing a gene into a host organism that does not have the gene such that the gene is expressed therein. Specific means for causing overexpression of a nonsymbiotic globin gene are not limited and any techniques known by persons skilled in the art can be employed. An example of a general method is a technique that involves ligating a nonsymbiotic globin gene downstream of an overexpression promoter so as to enable the expression of the gene in a correct reading frame and then introducing the thus constructed transformation vector into a nodulating plant, but the examples are not limited thereto. In this case, a nonsymbiotic globin gene is incorporated into a vector by, for example, excising a DNA fragment containing the nonsymbiotic globin gene using an appropriate restriction enzyme and then inserting in-frame and ligating the DNA fragment into an appropriate restriction enzyme site located downstream of an overexpression promoter in an expression vector containing the overexpression promoter. Alternatively, a DNA fragment previously prepared by ligating a nonsymbiotic globin gene downstream of an overexpression promoter may be incorporated into a vector. A genomic fragment of a nonsymbiotic globin gene ligated to an overexpression promoter may also be inserted into the genomic DNA of a nodulating plant via a process such as homologous recombination. As nodulating plants in which overexpression of nonsymbiotic globin genes is caused, any of the above nodulating plants (e.g., Lotus japonicus) can be appropriately used.

In the present invention, “overexpression promoter” means a promoter capable of causing strong expression (large amount expression) of a gene that has been ligated thereto in host plant cells. The overexpression promoter of the present invention may particularly be a nodule-specific promoter that enables nodule-specific expression. The overexpression promoter of the present invention may be either an inducible promoter or a constitutive promoter. A promoter is a DNA comprising an expression control region generally located on the 5′ upstream of a structural gene or a modified sequence thereof. In the present invention, any promoters appropriate for foreign gene expression in plant cells can be used as overexpression promoters. Preferred examples of such overexpression promoters to be used in the present invention include, but are not limited to, a cauliflower mosaic virus (CaMV) 35S promoter, a rice actin promoter, a modified 35S promoter, a tobacco PR1a promoter, and an Arabidopsis thaliana PR-1 promoter.

As a transformation vector for introduction of a nonsymbiotic globin gene, any gene introduction vector for plant cells can be used. For example, when an Agrobacterium method is employed, an Agrobacterium-derived plasmid vector (e.g., a Ti plasmid) or a binary vector is preferably used. Such transformation vector to be used in the present invention contain a nonsymbiotic globin gene and optionally an overexpression vector, and may further contain a selectable marker gene for facilitating selection of transformants, a reporter gene, a replication origin (e.g., Ti or Ri plasmid-derived replication origin) for use with a binary vector system, and the like. Examples of a selectable marker gene include drug resistance genes such as a Cefotax gene, a hygromycin resistance gene, a dihydrofolate reductase gene, an ampicillin resistance gene, a neomycin resistance gene, and a kanamycin resistance gene. Examples of a reporter gene include a green fluorescence protein gene (GFP) and luciferase genes (LUC and LUX). In the present invention, a “vector” encompasses so-called expression cassette. An “expression cassette” means a DNA fragment containing a promoter DNA sequence and the DNA sequence of a gene to be expressed, in which the DNA sequence of the gene is ligated to the promoter DNA sequence such that the gene can be expressed in plant cells. Such expression cassette may lack the ability of autonomous replication.

Furthermore, examples of a vector that contains an overexpression promoter in advance include pBI-based binary vectors (e.g., pKANNIBAL, IG121-Hm, pBI121, pBI101, pBI101.2, pBI101.3, and pCAMBIA1301).

The present invention also provides a vector comprising the above-mentioned nonsymbiotic globin gene ligated to an overexpression promoter. Such transformation vector can be very conveniently used because the vector can be introduced into any nodulating plants to enhance the nitrogen-fixing activity of nodules.

Any plant transformation methods that are broadly used for plants can be used as a method for introducing a transformation vector into a nodulating plant, including, but are not limited to, an Agrobacterium method, a particle gun method, an electroporation method, a polyethylene glycol (PEG) method, a microinjection method, and a protoplast fusion method. These plant transformation methods are described in ordinary textbooks such as “New Edition, Experimental Protocols for Model Plants, Genetic Techniques to Genomic Analysis” (under the supervision of Isao Shimamoto and Kiyotaka Okada, (2001) Shujunsha Co., Ltd.). Preferably, plant cells which have been subjected to the introduction of the transformation vector are selected by a method utilizing a selectable marker such as kanamycin resistance, and confirmed for the expression of the transgene through detection of a reporter protein or expression analysis of the transgene. Preferably, in addition to that, the plant bodies are regenerated by a conventional technique.

More specifically, in the case of the Agrobacterium method, for example, the method of Nagel et al is employed. First, a vector is introduced into Agrobacterium by electroporation. Next, plants are infected with the thus transformed Agrobacterium to introduce the gene of interest into the plants according to the method described in Plant Molecular Biology Manual (S. B. Gelvin et al., Academic Publishers); Thykaer, T. et al., Cell Biology, 2nd ed. (1998) pp. 518-525; Stiller, J., et al., J. Exp. Bot. (1997) 48, pp. 1357-1365; Ogar, P. et al., Plant Science (1996) 116 159-168; or Hiei Y. et al., Plant J. (1994) 6, 271-282. Plant bodies are then regenerated.

When the polyethylene glycol method is employed, first, cell walls may be removed by digesting them with enzymes to obtain protoplasts. A nonsymbiotic globin gene may be then introduced into the cells using polyethylene glycol and then the cells may be regenerated into plant bodies (Datta SK: In Gene Transfer To Plants (Potrykus I and Spangenberg, Eds) pp. 66-74 (1995)).

When the electroporation method is employed, first, cell walls may be removed by digesting them with an enzyme to obtain protoplasts. Electric pulses may be applied to the protoplasts to introduce a nonsymbiotic globin gene into cells and then the cells may be regenerated into plant bodies (Toki S, et al., Plant Physiol., 100: 1503 (1992)).

When the particle gun method is employed, plant bodies, plant organs, and plant tissues may be used intact. Alternatively, sections or protoplasts may be prepared therefrom and then used (Christou P, et al., Biotechnology 9: 957 (1991)). The prepared sample is bombarded with microparticles (each with a diameter between approximately 1 μm and 2 μm) of gold, tungsten, or the like that have been coated with a nonsymbiotic globin gene with high pressure gas using a gene transfer apparatus (e.g., PDS-1000 (BIO-RAD)) to introduce the nonsymbiotic globin gene into plant cells. Treatment conditions may vary depending on plant and sample used, and but in general, the bombardment is performed at a pressure between approximately 450 psi and 2000 psi and at a distance approximately between 4 cm and 12 cm from the target. Once the nonsymbiotic globin gene is introduced into the cells, the cells may be regenerated into plant bodies as described above.

In the thus transformed nodulating plant of the present invention, the nonsymbiotic globin gene may be expressed in an inserted form in the genomic DNA of the plant or may be expressed as an extragenomic DNA (e.g., in a form that is retained in a vector).

Expression of a nonsymbiotic globin gene in transformed plant cells or plant tissues (e.g., hairy roots, leaves, stems, and nodules) in which the overexpression of the nonsymbiotic globin gene has been caused as described above or in the regenerated plant bodies thereof is preferably confirmed by a conventional technique such as a Northern blotting method, a Southern blotting method, or a method on the expression of a reporter gene.

### 4. FORMATION OF NODULES IN PLANT BODIES AND DETERMINATION OF NITROGEN-FIXING ACTIVITY OF NODULES

Transformed nodulating plants in which overexpression of a nonsymbiotic globin gene has been caused according to the present invention can form nodules when they are grown under a given environment in which a symbiotic nitrogen-fixing bacterium is present. However, it is also possible to artificially cause nodulation through inoculation of such nodulating plant with a symbiotic nitrogen-fixing bacterium. Such nodulating plant can be inoculated with a symbiotic nitrogen-fixing bacterium according to a method well known by persons skilled in the art (e.g., see Higashi, S., Katahira, S, and Abe, M. Plant and Soil 81, pp. 91-99 (1984)).

The term “symbiotic nitrogen-fixing bacteria” used herein means microorganisms that are capable of living symbiotically with plants, forming nodules, fixing nitrogen into ammonia and supplying it to the host plants (that is, have nitrogen-fixing ability). Such symbiotic nitrogen-fixing bacteria include, when they are eubacteria, root nodule bacteria of the genera Rhizobium, Bradyrhizobium, Azorhizobium, Sinorhizobium, Mesorhizobium, and Allorhizobium, and when they are Actinomycetes, bacteria of the genus Frankia. Root nodule bacteria are known to infect leguminous plants and plants of the genus Parasponia of the family Ulmaceae. Bacteria of the genus Frankia are known to infect mainly trees of the family Betulaceae, Myricaceae, or the like. Some exceptions are also known for such symbiotic nitrogen-fixing bacterium-host plant relationship. Hence, a transformed nodulating plant may be inoculated with a symbiotic nitrogen-fixing bacterium capable of infecting the plant species to which the target plant belongs. For example, Lotus japonicus may be inoculated with Lotus japonicus root nodule bacteria (Mesorhizobium loti).

When a nodulating plant that has overexpressed a nonsymbiotic globin gene is inoculated with a symbiotic nitrogen-fixing bacterium to form nodules according to the present invention, nitrogen-fixing activity in the nodules is significantly enhanced. The nitrogen-fixing activity of such nodules is enhanced at least 2 times and preferably at least 3 times (e.g., 3 to 6 times) per unit weight of the nodules, compared with the level of such activity in a control plant (wild-type strain) of the same species that has not overexpressed the nonsymbiotic globin gene.

The nitrogen-fixing activity of nodules may be determined using any method for determining nitrogen-fixing activity that is known by persons skilled in the art. In the present invention, the nitrogen-fixing activity of nodules is preferably determined as the activity of reducing acetylene to ethylene (acetylene reduction activity: ARA activity) in an extract of the nodules. ARA activity can be expressed as the amount of ethylene generated per unit weight (e.g., 1 gram) of nodules and per unit time (e.g., 1 hour or 1 minute). Such ARA activity may be determined according to the Examples described below, for example. The nodulating plant that has overexpressed a nonsymbiotic globin gene according to the present invention exhibits ARA activity of between 10 nM/min/g and 100 nM/min/g and preferably of between 11 nM/min/g and 40 nM/min/g in nodules, but examples are not limited thereto.

The present invention further relates to a nodulating plant obtained as described above, which has overexpressed a nonsymbiotic globin gene and has nodules on its roots.

### 5. OTHER EMBODIMENTS

A nodulating plant that is produced by the method of the present invention forms nodules exhibiting high nitrogen-fixing activity. Hence, through cultivation of such plant, a large amount of nitrogen in atmosphere can be fixed under an environment for cultivation. Therefore, the present invention also provides a method for enhancing nitrogen-fixing efficiency upon plant cultivation. The expression “enhancing nitrogen-fixing efficiency upon plant cultivation” means to increase the nitrogen fixation level per predetermined cultivation area or per plant body under cultivation within a predetermined time period, compared with the nitrogen fixation level achieved by a non-transformed corresponding plant under the same environment. In the present invention, “cultivation” means to intentionally grow a relevant plant in a specific place or under specific environment. In the present invention, the expression “cultivation” encompasses agricultural cultivation, but agricultural work (tilling, seeding, planting seedlings, thinning out, disinfection, pruning, thinning, and harvest) is not always required. Examples of cultivation in the present invention include, but are not limited thereto, cultivation of crops or plants for gardening, landscaping, gardening, planting for greening degraded lands or seashores, planting for fertilization of oligotrophic soil, and planting for improving the quality of soil such as saline soil or dry soil.

Through enhancement of nitrogen-fixing efficiency upon plant cultivation, a nodulating plant that is produced by the method of the present invention can increase the amount of nitrogen to be fixed from atmosphere under a given environment, can increase the concentration of fixed nitrogen in the relevant plant tissues, and thus can increase the yield or the growth amount of the nodulating plant. Furthermore, in the long term, cultivation of the nodulating plant of the present invention enables fertilization of soil through enhancement of the amount of nitrogen in soil and increase of the yields of other plants using such soil. Moreover, cultivation of the nodulating plant of the present invention enables effective greening of oligotrophic soil, saline soil, dry soil, and the like.

### EXAMPLES

The present invention is further illustrated with reference to the following examples. However, these examples do not limit the technical scope of the present invention.

### Example 1

### Isolation and Identification of Lotus japonicus Nonsymbiotic Globin Gene (LjHb1)

A nonsymbiotic globin gene (LjHb1) of Lotus japonicus was isolated through screening of a Lotus japonicus genomic library (Sato et al. (2000) DNA Res. 8: pp. 311-318) by means of the PCR method. Primers used for screening were designed based on the sequence of a nonsymbiotic globin gene homolog (clone name: AV413959, DDBJ/EMBL/GenBank accession No. AB238220) that is present in the EST library of Lotus japonicus. The primer sequences are shown as follows:

LjHb1F1: 5′-TTCTCACTTCACTTCCATCGC-3′
(SEQ ID NO: 4, forward primer);
LjHb1F2: 5′-TTGGTCAAGTCATGGAGCG-3′
(SEQ ID NO: 5, forward primer);
LjHb1R1: 5′-TCACAGTGACTTTTCCAGCG-3′
(SEQ ID NO: 6, reverse primer);
and
LjHb1R2: 5′-AGACAGACATGGCATGAGGC-3′
(SEQ ID NO: 7, reverse primer).

PCR for amplification of the LjHb1 gene was performed under the following reaction conditions using GeneAmp® PCR System 9700 (Applied Biosystems): 30 cycles of 94° C. for 30 seconds, 55° C. for 30 seconds, and 72° C. for 30 seconds.

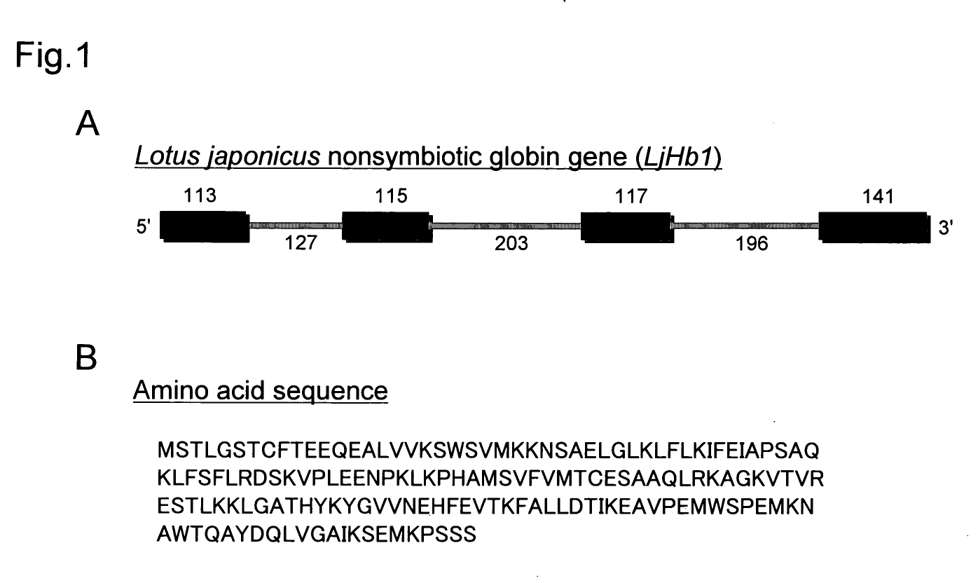

As a result of the screening of Lotus japonicus genomic library, the LjHb1 gene present in a TAC clone (LjTOI01) was identified. The LjHb1 gene had a full length of 1012 bp spanning from the initiation codon to the termination codon and was a gene encoding 161 amino acid residues, as revealed by sequence analysis of the LjHb1 gene in the Lotus japonicus genome. The genomic structure of the Lotus japonicus nonsymbiotic globin gene (LjHb1) and the amino acid sequence encoded by the gene are shown in FIG. 1. The LjHb1 gene had a structure containing four exons and three introns, which is a common structure among plant globin genes, and was present on chromosome 3 among the six chromosomes of Lotus japonicus.

### Example 2

### Expression Analysis of the Nonsymbiotic Globin Gene (LjHb1)

The expression analysis of LjHb1 was performed by means of RT-PCR in two experimental series in which the expression in different tissues and the expression under stress conditions were examined, respectively. In the experimental series involving the examination of the expression in different tissues, four types of tissue sample of Lotus japonicus (grown individual plants on week 6 after germination) were used: 1) leaves; 2) stems; 3) roots; and 4) nodules. In the experimental series involving the examination of the expression under stress conditions, the four types of sample used herein were: 1) untreated (control) sample; 2) sucrose-added sample; 3) low temperature-treated sample; and 4) low oxygen-treated sample. Primers LjHb1F1 and LjHb1R2 that had been used in Example 1 were used for the RT-PCR. A one-step RT-PCR kit (QIAGEN) was used for the reverse transcription and the amplification of transcription products thereof. The expression levels of the LjHb1 gene were determined by electrophoresis. Electrophoretic photographs were subjected to imaging. LjHb1 expression levels were expressed as relative values based on the band intensities.

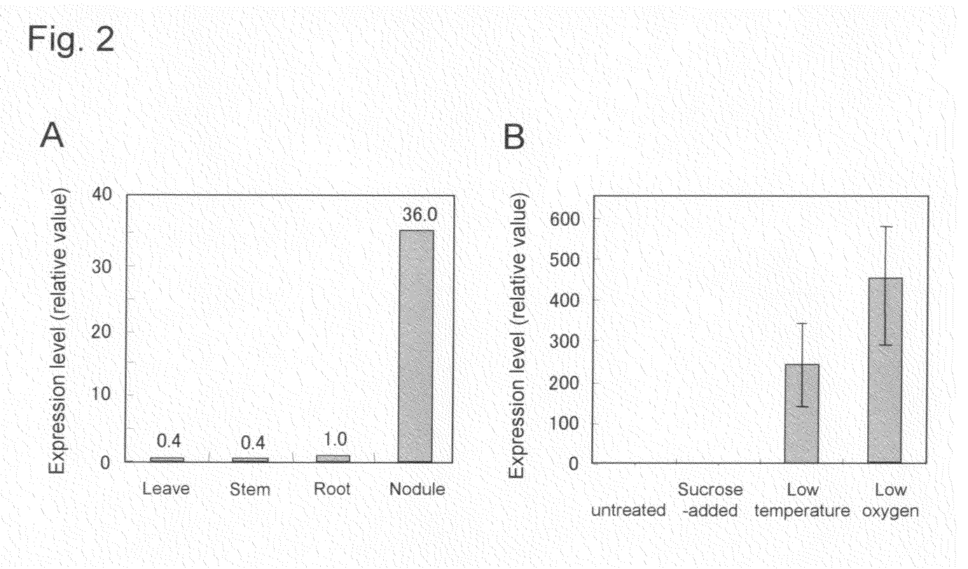

FIG. 2A shows the experimental results of examining the expression in different tissues. It was demonstrated that LjHb1 is expressed in various organs of grown individual plants. Expression levels (relative values) were: 0.4 in the leaves; 0.4 in the stems; 1.0 in the roots; and 36.0 in the nodules. Thus, LjHb1 was particularly strongly expressed in the nodule tissues. Furthermore, FIG. 2B shows the experimental results of examining the expression under stress conditions. It was demonstrated that LjHb1 is also strongly expressed via stress treatments such as low temperature treatment (see, FIG. 2B; expression level: approximately 250) and low oxygen treatment (FIG. 2B; expression level: approximately 450), in terms of relative expression level compared with an untreated sample.

### Example 3

### Construction of Transformation Vectors

For the purpose of producing transformed Lotus japonicus plant bodies and transformed hairy roots which overexpress LjHb1, a gene construct comprising LjHb1 cDNA ligated to a strong promoter was prepared. Further, for construction of vectors to be used for transformation, plasmids pKANNIBAL (Wesley et al. 2001 the plant Journal 27, 581-590), pHKN29 (Kumagai and Kouchi, 2003 MPMI 16(8), 663-668) and pIG121-Hm (Ohta et al., 1990, Plant Cell Physiol., 31, 805-813) were used.

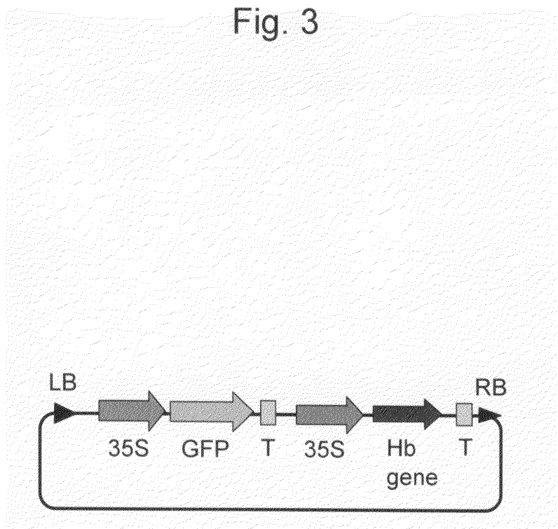

Total RNA was extracted according to a conventional technique from Lotus japonicus nodules, and then cDNA was synthesized by RT-PCR. Full-length LjHb1 cDNA was cloned through PCR using the resulting cDNA as a template. In particular, the obtained full-length LjHb1 cDNA (SEQ ID NO: 1) was ligated downstream of the cauliflower mosaic virus 35S promoter in pKANNIBAL. Further, 35S-LjHb1 cDNA fragment was excised from the resulting vector and then ligated downstream of the GFP region in pHKN29 to produce plasmid vector pR35SLjHb1 (FIG. 3). This pR35SLjHb1 was used for induction of transformed hairy roots as described below.

Furthermore, full-length LjHb1 cDNA was ligated downstream of the 35S promoter in pIG12I-Hm to produce a plasmid vector pT35SljLb1. This pT35SljLb1 was used for producing transformed Lotus japonicus plant bodies as described below.

### Example 4

### Production of Transformed Hairy Roots and Confirmation of Gene Introduction and Gene Expression

Transformed Lotus japonicus hairy roots into which LjHb1 gene had been introduced were produced by a hairy-root-inducing transformation system mediated by Agrobacterium rhizogenes using the plasmid vectors constructed in Example 3.

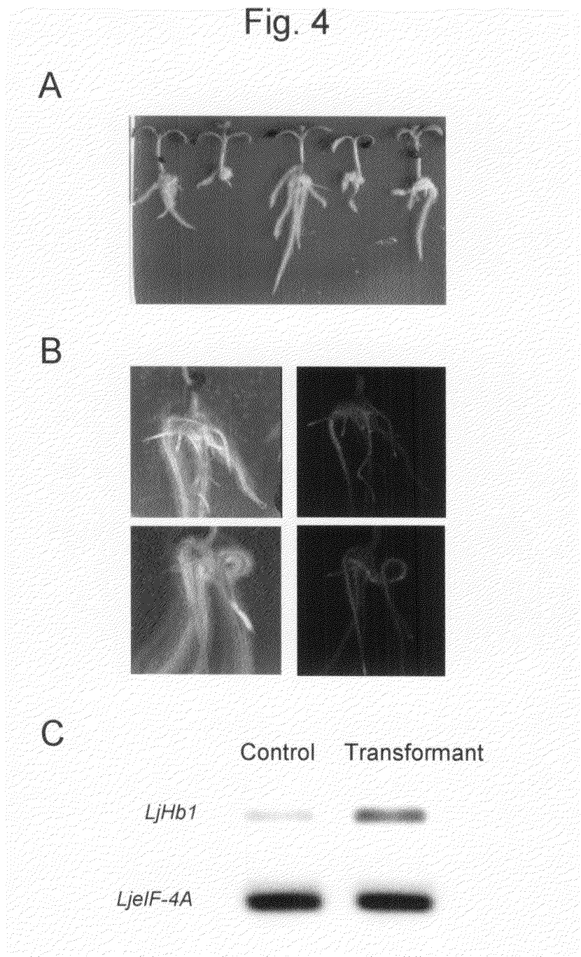

First, the vector pR35SLjHb1 that enables overexpression of LjHb1 constructed in Example 3 was directly introduced into Agrobacterium rhizogenes LBA1334 (provided by Dr. Clara Diaz (Institute Molecular Plant Science, Leiden University)) by electroporation. Lotus japonicus seedlings on day 5 after seeding, excised at the hypocotyl portions, were inoculated with a bacterial cell suspension of the Agrobacterium rhizogenes harboring pR35SLjHb1. Subsequently, the seedlings were placed on sterilized filter papers and then co-cultivated in the co-cultivation medium (1/10 B5, 0.5 μg/ml BAP, 0.05 μg/ml NAA, 5 mM MES (pH 5.2), and 20 μg/ml acetosyringone) for 5 days. After the completion of co-cultivation, the seedlings were placed in GamborgB5 medium (produced by Nihon Pharmaceutical Co., Ltd., and marketed by Wako Pure Chemical Industries, Ltd.) supplemented with antibiotic cefotax (200 μg/ml; Chugai Pharmaceutical Co., Ltd.) so that hairy roots were induced. FIG. 4A shows the thus induced hairy roots. In addition, FIG. 4A shows various samples differing in terms of growth stage or period of hairy root development.

The presence or the absence of the transgene LjHb1 in hairy roots was confirmed through GFP fluorescence detection and detection of a fusion gene of 35S promoter and LjHb1 via PCR amplification. In hairy roots into which the LjHb1 gene had been introduced, GFP was produced and thereby green fluorescence was observed (FIG. 4B). In FIG. 4B, the left (upper and lower) photographs are bright-field images observed and the right (upper and lower) photographs are dark-field images observed. GFP fluorescence was clearly observed in roots as a result of overexpressing the symbiotic globin gene.

Furthermore, total RNA was extracted according to a conventional technique from the transformed hairy roots, and then RT-PCR was performed so that the expression of the transgene LjHb1 was confirmed. In addition, Lotus japonicus hairy roots derived from a wild-type strain were used as controls. FIG. 4C shows the results. As shown in FIG. 4C, the LjeIF-4A gene which is known to be expressed at the same level in any Lotus japonicus tissues and at any stages was used as an indicator for demonstrating that the RNA amount used for the causing of the gene expression was the same in both test and control samples.

As demonstrated by such results, in hairy roots into which the LjHb1 gene had been introduced, LjHb1 gene expression was induced in an amount approximately more than 100 times greater than that in the hairy roots of the wild-type strain into which LjHb1 gene had not been introduced.

### Example 5

### Nodulation of Transformed Hairy Roots and Measurement of Nitrogen-Fixing Activity of the Nodules

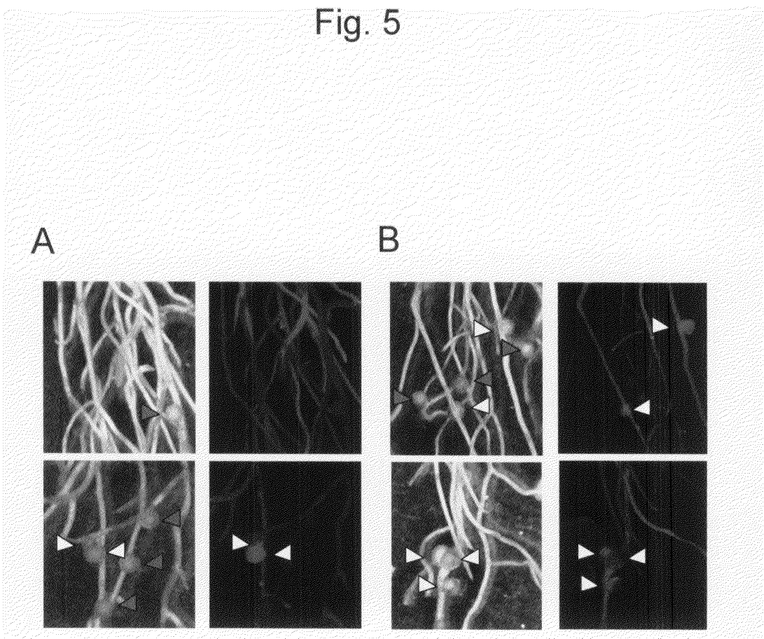

The plants were induced to develop hairy roots and LjHb1 gene introduction therein was confirmed in Example 4. The plants were transplanted to culture soil (vermiculite:pearlite=4:1). Fahraeus medium (Fahraeus, (1957) J. Gen. Microbiol. 16(2) 374-381) supplemented with KNO₃ up to a final concentration of 1 mM was given to the plants, and the plants were grown for 1 week. Subsequently, the plants were transplanted to a fresh culture soil and then the hairy roots were inoculated with Lotus japonicus root nodule bacteria (Mesorhizobium loti MAFF 303099) at a concentration of 1×10⁷ cells/ml. After inoculation with the root nodule bacteria, medium containing no nitrogen source was given thereto, and then the plants were further grown for 4 weeks. The transformed plants were kept under the growth conditions as in a plant growth chamber, with a 16 hours light/8 hours dark cycle, at 25° C. to 26° C. 4 weeks later, nodules were formed on the hairy roots (FIG. 5).

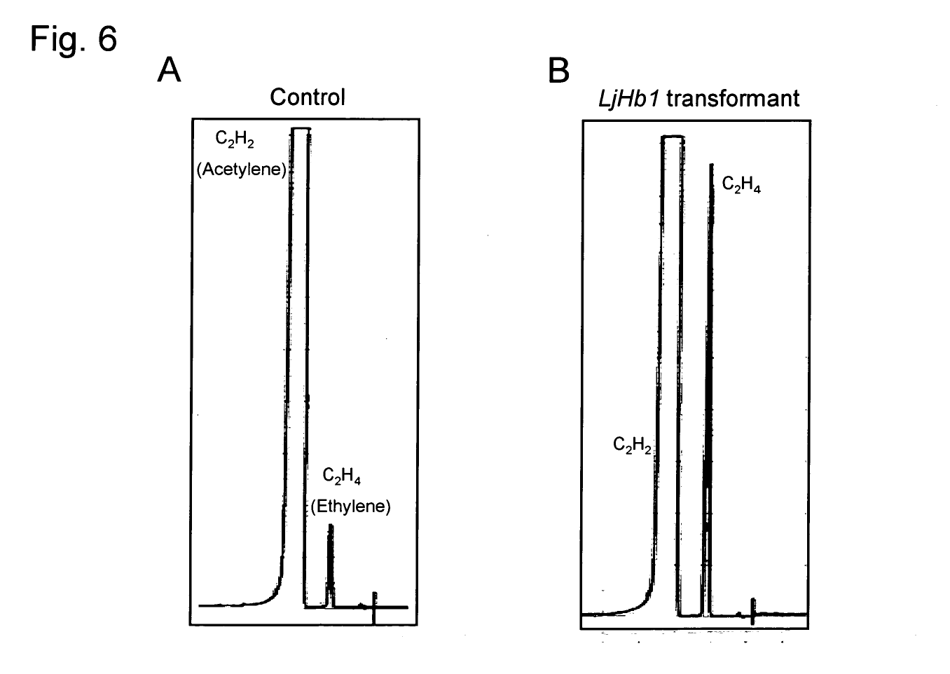

Next, the nitrogen-fixing activity of the nodules formed on the hairy roots was determined as activity of reducing acetylene into ethylene (Acetylene Reduction Activity; ARA). Specifically, first, nodules collected from the hairy roots were put into a 15-cm test tube and then the test tube was sealed with a rubber cap. Air within the test tube was sufficiently aspirated off using an aspirator and then the test tube was filled with an acetylene gas. After 2 hours of incubation at room temperature, the gas within the test tube was collected and then the amount of generated ethylene was measured by gas chromatography. FIG. 6 shows the measurement results.

As in the measurement results shown in FIG. 6, ARA activity per unit weight of nodules (i.e., per gram weight of nodules) formed on the hairy roots into which LjHb1 had been introduced and overexpressed was calculated to be 11.15 nM/min/g [1.45×21.5−0.5=30.67 (nM/g); 30.67 (nM)/2.75 minutes=approximately 11.15 nM/min/g]. On the other hand, as a control, ARA activity per unit weight of nodules (per gram weight of nodules) formed on the hairy roots into which no LjHb1 had been introduced was calculated to be 2.12 nM/min/g [1.45×4−0.5=5.3 (nM/g); 5.3 (nM)/2.5 minutes=2.12 nM/min/g]. It was shown that the nitrogen-fixing activity of nodules formed on the hairy roots (transformants) into which LjHb1 had been introduced and overexpressed was 5 times or more greater than that of the control.

### Example 6

### Production of Transformed Plant Bodies

1) Infection of Lotus japonicus with Agrobacterium tumefaciens (A. tumefaciens)

Each hypocotyl was excised from a Lotus japonicus seedling on day 5 after seeding by cutting at a site immediately below the cotyledon and at a boundary of the root. Meanwhile, pT35SljLb1 constructed in Example 3 was directly introduced into Agrobacterium tumefaciens (A. tumefaciens) EHA105 (provided by Dr. Kenzo Nakamura at the Nagoya University) by electroporation. Acetosyringone was added at a final concentration of 100 μM to a bacterial cell suspension (1.0×10⁷ cells/ml, OD₆₀₀=0.10×0.15) of the resulting Agrobacterium tumefaciens EHA105 harboring pT35SljLb1. The excised hypocotyl was immersed in the suspension solution. The hypocotyl was sliced into sections with a thickness of approximately 5 mm in the solution. The sections were continuously immersed in the bacterial cell suspension for 30 minutes, so that the sections were infected with Agrobacterium. After infection, the sections were placed on sterilized filter paper and then co-cultivated at 25° C. for 3 to 5 days in the co-cultivation medium (1/10 B5, 0.5 μg/ml BAP, 0.05 μg/ml NAA, 5 mM MES (pH 5.2), and 20 μg/ml acetosyringone).

### 2) Callus Induction

Co-cultivated sections were transferred onto a callus medium (1×B5, 2% sucrose, 0.5 μg/ml BAP, 0.05 μg/ml NAA, 10 mM NH₄, and 0.3% phytagel) supplemented with antibiotic cefotax (250 μg/ml) for sterilization and antibiotic hygromycin B for selection of the transformant. The sections were cultured at 25° C. with a 14 hours light/10 hours dark cycle for 5 weeks. Transplantation of sections was performed every 1 to 2 weeks.

3) Induction of Shoots from Calli

After 5 weeks of culture in the above callus medium, the sections were transferred onto a shoot induction medium (1×B5, 2% sucrose, 0.5 μg/ml BAP, 0.05 μg/ml NAA, 10 mM NH₄, and 0.3% phytagel). The sections were cultured at 25° C. with a 14 hours light (6100 lux)/10 hours dark cycle for 2 weeks. Subsequently, the calli formed from the sections were transplanted in shoot induction medium not supplemented with hygromycin B. The calli were cultured under culture conditions that were the same as those described above for 3 weeks. Transplantation of calli was performed every 1 to 2 weeks.

### 4) Shoot Elongation

Calli were transferred onto a shoot elongation medium (1×B5, 2% sucrose, 0.2 μg/ml BAP, and 0.3% phytagel) and then cultured at 25° C. with a 14 hours light (6100 lux)/10 hours dark cycle for 3 weeks. Transplantation of calli was performed every 1 to 2 weeks. Subsequently, calli were transplanted onto shoot elongation medium containing no plant hormone. The calli were then cultured under culture conditions similar to those described above for 2 to 3 weeks, thereby promoting shoot elongation.

### 5) Induction and Elongation of Roots

5 mm or longer shoots generated from the calli placed on the shoot elongation medium were cut from each shoot base portion using a razor. The shoot base portions inserted lengthwise in a root induction medium (1/2 B5, 1% sucrose, 0.5 μg/ml NAA, and 0.4% phytagel), and the shoots were cultured with a 14 hours light (6100 lux)/10 hours dark cycle for 1 weeks or more. Subsequently, the shoots with bloating cut areas were inserted into a root elongation medium (1/2 B5 and 1% sucrose). The shoots were then cultured under culture conditions similar to those described above for 2 to 3 weeks, thereby promoting root elongation.

### 6) Cultivation of Transformed Plant Bodies

Plant bodies were obtained through root elongation as described above. The plant bodies were extracted from the medium and then gel that had adhered to the roots was thoroughly washed off in water. The plant bodies were transplanted onto vermiculite impregnated with commercial B5 medium (Wako Pure Chemical Industries, Ltd.) diluted 1:10. The plant bodies were then cultivated with a 14 hours light (6100 lux)/10 hours dark cycle. The thus cultivated Lotus japonicus plant bodies produced seeds, and the seeds were harvested. Subsequently, the seeds were seeded in Power soil (culture soil for gardening; Kureha Chemical Co.) for cultivation.

Whether or not the LjHb1 gene had been successfully introduced into the thus grown plant bodies was confirmed by detecting the fusion gene of a 35S promoter and LjHb1 through PCR amplification thereof. Furthermore, increases in the expression levels of the LjHb1 gene were confirmed by RT-PCR.

### Example 7

### Nodulation and Nitrogen-Fixing Activity of Transformed Lotus japonicus Plant Bodies

Transformed Lotus japonicus plant bodies were produced and the introduction and expression of the LjHb1 gene into the plant bodies were confirmed as in Example 6 described above. The transformed plant bodies were transplanted into culture soil (vermiculite:pearlite=4:1) for cultivation. The plant bodies were inoculated with Lotus japonicus root nodule bacteria (Mesorhizobium loti MAFF 303099) at a concentration of 1×10⁷ cells/ml. After inoculation with the root nodule bacteria, the transformed plant bodies were cultivated in a plant growth chamber, with a 16 hours light (6100 lux)/8 hours dark cycle at 25° C. to 26° C. for 4 weeks. After 4 weeks of cultivation, the formed nodules were collected from the plant bodies and then ARA activity was determined in a manner similar to that in Example 5. As a result of determination, the ARA activity per unit weight of nodules (per gram weight of nodules) formed by transformed Lotus japonicus plant bodies overexpressing LjHb1 was calculated to be 17.21 nM/min/g. As a control, the ARA activity per unit weight (per gram weight of nodules) of nodules formed on Lotus japonicus (general Lotus japonicus, wild-type) into which LjHb1 had not been introduced was calculated to be 5.05 nM/min/g.

In addition, the average number of nodules formed on each plant body obtained in this example was 7 in both transformants and non-transformants. No particular differences were observed in the appearance of nodules, such as size and color.

### Example 8

### Comparison of the Affinity for Nitrogen Monoxide of Lotus japonicus Nonsymbiotic Globin with that of Lotus japonicus Symbiotic Globin

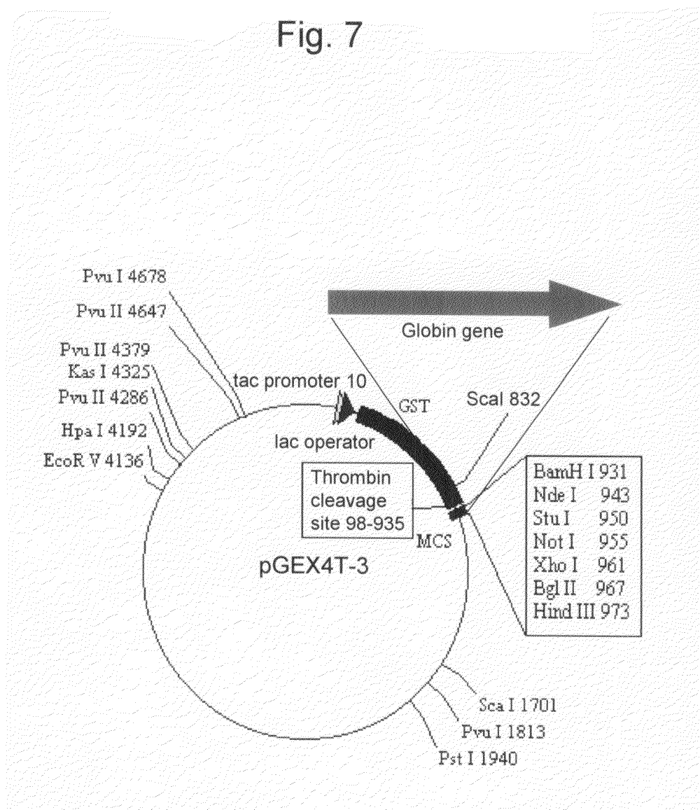

The full-length LjHb1 cDNA (the sequence spanning from the initiation codon to the termination codon thereof is shown in SEQ ID NO: 1, which encodes the amino acid sequence of SEQ ID NO: 2) obtained in Example 3 was cloned into a protein expression vector pGEX4T-3 (Amersham Pharmacia Biotech) according to a conventional technique (FIG. 7). The vector was then introduced into Escherichia coli to obtain transformants. For preparation of a control sample, a Lotus japonicus symbiotic globin gene was similarly cloned into an expression vector pGEX4T-3 (FIG. 7), and the vector was then introduced into Escherichia coli to obtain transformants. The thus obtained Escherichia coli transformants were cultured for inducing gene expression. Hence, soluble active nonsymbiotic globin could be recombinantly produced within bacterial cells of Escherichia coli in large amounts. Subsequently, Escherichia coli cells that had expressed the nonsymbiotic globin gene were collected according to a conventional technique. After disruption of E. coli cells, protein purification was performed, so that active nonsymbiotic hemoglobin could be obtained. The globin to be obtained through disruption of Escherichia coli cells was associated with heme from Escherichia coli and thus isolated as a form having hemoglobin activity, because heme is also supplied in Escherichia coli into which the nonsymbiotic globin gene had been introduced.

Subsequently, nitrogen monoxide was mixed with the thus obtained symbiotic hemoglobin and then absorbance was measured over time. FIG. 8 shows absorbance spectra obtained at wavelengths between 500 nm and 600 nm at 0 minutes, 5 minutes, 15 minutes, and 30 minutes after the initiation of mixing with nitrogen monoxide. As shown in FIG. 8, as the time of mixing with nitrogen monoxide lengthens, two peaks at 540 nm and 575 nm progressively disappeared. Particularly in the case of nonsymbiotic hemoglobin, the 575-nm peak disappeared at a faster rate and almost completely disappeared at 15 minutes after the initiation of mixing. On the other hand, in the case of symbiotic hemoglobin, the 540-nm peak was not dramatically decreased. Even at 30 minutes after the initiation of mixing, the 575-nm peak remained, although it was weak, so that two peaks were still observed.

### Example 9

### Isolation and Identification of Alnus firma Nonsymbiotic Globin Gene (AfHb1)

A nonsymbiotic globin gene (AfHb1) of Alnus firma was isolated by screening of an Alnus firma nodule cDNA library (Sasakura, F. et al., “A class 1 hemoglobin gene from Alnus firma functions in symbiotic and nonsymbiotic tissues to detoxify nitric oxide.” Mol. Plant. Microbe. Interact. (2006) 19(4) in press) using a DNA fragment of the Lotus japonicus nonsymbiotic globin gene LjHb1 as a probe.

The probe for screening was designed based on the sequence of the Lotus japonicus nonsymbiotic globin gene LjHb1 (clone name: AV413959, DDBJ/EMBL/GenBank accession No. AB238220). The probe was obtained by PCR amplification from the genomic DNA of Lotus japonicus using the primers LjHb1F1 (5′-TTCTCACTTCACTTCCATCGC-3′: SEQ ID NO: 4) and LjHb1R1 (5′-TCACAGTGACTTTTCCAGCG-3′: SEQ ID NO: 6) used in Example 1. PCR for amplifying a LjHb1 fragment to be used as a probe was performed using GeneAmp® PCR System 9700 (Applied Biosystems) under conditions consisting of 30 cycles of 94° C. for 30 seconds, 55° C. for 30 seconds, and 72° C. for 30 seconds.

As a result of such screening of the Alnus firma nodule cDNA library, the AfHb1 gene was identified. As a result of nucleotide sequence analysis, the AfHb1 gene was found to be a gene comprising a sequence of 483 bp in length spanning from the initiation codon to the termination codon in the cDNA and encoding 160 amino acids. The nucleotide sequence spanning from the initiation codon to the termination codon of cDNA sequence of AfHb1 (DDBJ/EMBL/GenBank accession No. AB221344) is shown in SEQ ID NO: 8. The amino acid sequence encoded by the nucleotide sequence is shown in SEQ ID NO: 9. Moreover, the genomic DNA sequence (from the initiation codon to the termination codon) of the Alnus firma AfHb1 gene is shown in SEQ ID NO: 10.

### Example 10

### Expression Analysis of Alnus firma Nonsymbiotic Globin Gene (AfHb1)

The expression analysis of AfHb1 was performed by RT-PCR using mRNAs extracted according to a conventional technique from the tissues of various organs of Alnus firma as samples. The following AfHb1F1 and AfHb1R3 primers were used for the RT-PCR. A One-Step RT-PCR kit (QIAGEN) was used for the reverse transcription and the amplification of transcription products thereof. The following AfHb1F1 and AfHb1R3 primers were also used for amplification of the reverse transcription products. The primer sequences used herein are shown as follows:

AfHb1F1: 5′-GCTGCTATCAAATCTGCAAT-3′
(SEQ ID NO: 11, forward primer)
and
AfHb1R3: 5′-GGGGGGCTGTGATTTTAG-3′
(SEQ ID NO: 12, reverse primer).

The thus obtained amplified products were electrophoresed and then electrophoretic photographs obtained were subjected to imaging. The expression level of the AfHb1 gene in each tissue was determined based on the band intensity.

The results of the expression analysis by means of RT-PCR revealed that the AfHb1 gene had been expressed in various organs of the grown plants of Alnus firma. In particular, the AfHb1 gene had been strongly expressed in nodule tissues. As with the Lotus japonicus LjHb1 gene, it was revealed that the expression of the AfHb1 gene was also strongly induced by stress treatment such as low temperature treatment.

### Example 11

### Construction of a Transformation Vector

Production of transformed Lotus japonicus overexpressing the AfHb1 gene was attempted to elucidate the functions of the AfHb1 gene. For vector construction for transformation, plasmids pKANNIBAL (Wesley et al., 2001, The Plant Journal 27, 581-590) and pHKN29 (Kumagai and Kouchi. 2003 MPMI 16(8), 663-668) were used.

First, cDNA was synthesized by reverse transcription from total RNA extracted from Alnus firma nodules. Full-length AfHb1 cDNA was cloned by means of PCR using the synthesized cDNA as a template. The thus obtained AfHb1 cDNA was ligated downstream of the cauliflower mosaic virus 35S promoter in pKANNIBAL. Moreover, the 35S-AfHb1 cDNA fragment was excised from that and then ligated downstream of GFP region in pHKN29, thereby finally preparing a transformation vector pAfHb1S.

### Example 12

### Production of Transformed Hairy Roots

A hairy-root-inducing transformation system mediated by Agrobacterium rhizogenes was employed for transformation of Lotus japonicus with AfHb1. The transformation method is based on the principle of co-transfection, by which hairy roots are induced and transformed by Agrobacterium rhizogenes. Transformed hairy roots in this example were produced according to the method of Example 3.

The transformation vector pAfHb1S that enables overexpression of the AfHb1 gene constructed in Example 11 was directly introduced into Agrobacterium rhizogenes LBA1334 by electroporation. Lotus japonicus seedlings on day 5 after seeding, which had been excised at the hypocotyl portions, were infected through inoculation with a bacterial cell suspension of the Agrobacterium rhizogenes LBA1334 in which the vector pAfHb1S had been introduced. Subsequently, the seedlings were placed on sterilized filter paper and then co-cultivated in co-cultivation medium for 5 days. After the completion of co-cultivation, the seedlings were placed on agar medium of Gamborg B5 medium (produced by Nihon Pharmaceutical Co., Ltd. and marketed by Wako Pure Chemical Industries, Ltd.) supplemented with antibiotic cefotax (200 μg/ml; Chugai Pharmaceutical Co., Ltd.), so that hairy roots were induced.

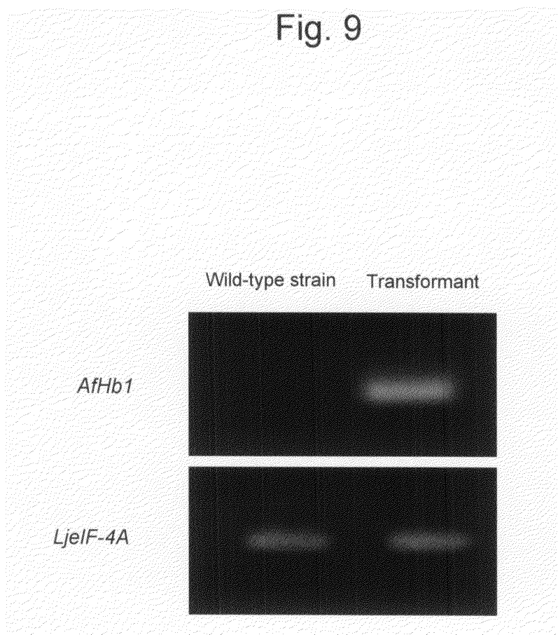

The presence or the absence of transgene AfHb1 in hairy roots was confirmed through GFP fluorescence detection and RT-PCR, in a manner similar to that in Example 4. As a result, in all the individual Lotus japonicus plants that had been infected with Agrobacterium rhizogenes into which AfHb1 had been introduced, hairy roots emitting GFP fluorescence were induced. On the other hand, as a result of confirmation by RT-PCR, AfHb1 gene expression was induced in hairy roots into which AfHb1 had been introduced (transformants) at an expression level of approximately more than 100 times greater than that in wild-type strain hairy roots into which AfHb1 had not been introduced. FIG. 9 shows the results of confirming AfHb1 gene expression using RT-PCR. In FIG. 9, LjeIF-4A is a positive control.

### Example 13

### Analysis of the Phenotypes of Individual Transformed Plants

It was confirmed in Example 12 that hairy roots had undergone transformation with the AfHb1 gene. Plants having such hairy roots were transplanted to culture soil (vermiculite:pearlite=4:1). Fahraeus medium (Fahraeus, (1957) J. Gen. Microbiol. 16(2) 374-381) supplemented with KNO₃ up to a final concentration of 1 mM was given to the plants and then the plants were grown for 1 week. Subsequently, the plants were transplanted to a fresh culture soil, and the hairy roots were inoculated with Lotus japonicus root nodule bacteria (Mesorhizobium loti MAFF 303099) at a concentration of 1×10⁷ cells/ml. After inoculation with the root nodule bacteria, Fahraeus medium containing no nitrogen source was given thereto, and then the plants were further grown for 4 weeks. The transformed plants were kept under the growth conditions as in a plant growth chamber, with a 16 hours light/8 hours dark cycle, at 25° C. to 26° C. Nodules were formed on the hairy roots of the grown plant bodies. The average number of nodules formed per each plant body was 9 in both transformants and non-transformants. No particular differences were observed among transformants and non-transformants in terms of appearance of nodules, such as size and color.

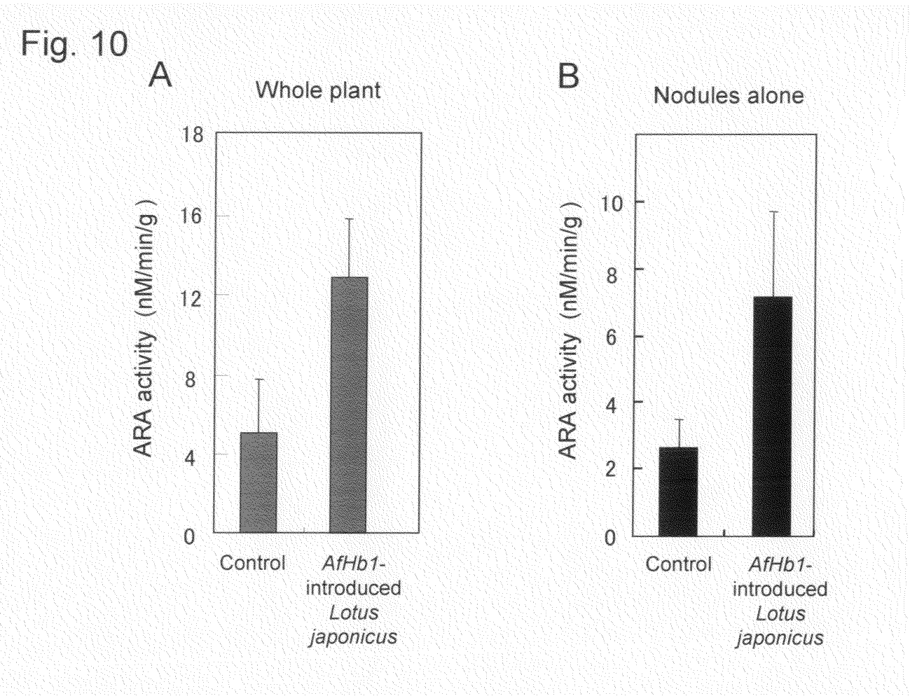

Next, the nitrogen-fixing activity of the nodules formed on the hairy roots was determined as one of phenotypes of the transformants. The nitrogen-fixing activity of nodules was determined as activity of reducing acetylene into ethylene (Acetylene Reduction Activity; ARA) according to the method described in Example 5. The results are shown in FIG. 10. In FIG. 10, “control” indicates the results obtained using, as a sample, Lotus japonicus into which the plasmid pHKN29 that does not contain the AfHb1 gene had been introduced.

As a result of determination, nodules formed on hairy roots into which AfHb1 had been introduced and overexpressed, exhibited nitrogen-fixing activity 3 to 5 times greater per unit weight of nodules compared with that of nodules formed on hairy roots of the wild-type strain Lotus japonicus into which AfHb1 had not been introduced. As shown in FIG. 10, nodules formed on hairy roots into which the overexpression of AfHb1 had been caused exhibited ARA activity (average) of 7.2 nM/min/g per unit weight. Determination was also performed for the whole Lotus japonicus plant bodies into which AfHb1 had been introduced, and the ARA activity (average) per unit weight was shown to be 13 nM/min/g. On the other hand, as a control, nodules formed on wild-type strain hairy roots into which AfHb1 had not been introduced exhibited ARA activity (average) of 2.6 nM/min/g per unit weight. The whole wild-type strain plant bodies into which AfHb1 had not been introduced exhibited ARA activity (average) of 5 nM/min/g per unit weight.

Based on the above results, it was demonstrated that the Lotus japonicus into which the Alnus firma nonsymbiotic globin gene, AfHb1, had been introduced also exhibits a significantly improved nitrogen-fixing activity of nodules formed on the hairy roots.

### INDUSTRIAL APPLICABILITY

According to the method for producing a nodulating plant of the present invention, a nodulating plant with nodules having markedly improved nitrogen-fixing activity can be obtained. Further, the method for enhancing nitrogen fixation levels upon plant cultivation through cultivation of the nodulating plant of the present invention can be used for increasing the yield or the growth amount of such nodulating plant or fertilizing soil by increasing the nitrogen level in soil and greening degraded lands and the like.

All publications, patents, and patent applications cited herein are incorporated herein by reference in their entirety.

### Sequence Listing Free Text

The sequences of SEQ ID NOs: 4 to 7, 11, and 12 represent primers.

1. A method for producing a nodulating plant capable of forming nodules having enhanced nitrogen-fixing activity, comprising causing overexpression of a nonsymbiotic globin gene in a nodulating plant.

2. The method according to claim 1, wherein said nonsymbiotic globin gene comprises a DNA selected from the group consisting of the following (a) to (e):
(a) a DNA comprising the nucleotide sequence shown in SEQ ID NO: 1 or 8;
(b) a DNA which hybridizes under stringent conditions to a DNA comprising a nucleotide sequence complementary to the nucleotide sequence shown in SEQ ID NO: 1 or 8 and encodes a protein having nonsymbiotic globin activity;
(c) a DNA encoding a protein comprising the amino acid sequence shown in SEQ ID NO: 2 or 9;
(d) a DNA encoding a protein that comprises an amino acid sequence derived from the amino acid sequence shown in SEQ ID NO: 2 or 9 by deletion, substitution, or addition of 1 to 50 amino acids and that has nonsymbiotic globin activity; and
(e) a DNA comprising the nucleotide sequence shown in SEQ ID NO: 3 or 10.

3. The method according to claim 1 or 2, wherein the overexpression of the nonsymbiotic globin gene is caused by introducing the nonsymbiotic globin gene ligated to an overexpression promoter into a nodulating plant.

4. The method according to claim 1, wherein said enhanced nitrogen-fixing activity is at least 3 times greater than that of a wild-type strain.

5. The method according to claim 1, wherein said nodules having enhanced nitrogen-fixing activity exhibit nitrogen-fixing activity of 10 nM/min/g to 100 nM/min/g.

6. The method according to claim 1, wherein said nodulating plant is a leguminous plant.

7. The method according to claim 1, further comprising inoculating the nodulating plant which has overexpressed the nonsymbiotic globin gene with a symbiotic nitrogen-fixing bacterium.

8. A nodulating plant which is produced by the method according to claim 1, wherein said nodulating plant is capable of forming nodules having enhanced nitrogen-fixing activity.

9. The nodulating plant according to claim 8, which has nodules on its roots.

10. A vector for enhancing nitrogen-fixing activity of nodules, comprising a nonsymbiotic globin gene ligated to an overexpression promoter.

11. The vector according to claim 10, wherein said nonsymbiotic globin gene is derived from a leguminous plant or a nonleguminous nodulating plant.

12. The vector according to claim 10 or 11, wherein said nonsymbiotic globin gene comprises a DNA selected from the group consisting of the following (a) to (e):
(a) a DNA comprising the nucleotide sequence shown in SEQ ID NO: 1 or 8;
(b) a DNA which hybridizes under stringent conditions to a DNA comprising a nucleotide sequence complementary to the nucleotide sequence shown in SEQ ID NO: 1 or 8 and encodes a protein having nonsymbiotic globin activity;
(c) a DNA encoding a protein comprising the amino acid sequence shown in SEQ ID NO: 2 or 9;
(d) a DNA encoding a protein that comprises an amino acid sequence derived from the amino acid sequence shown in SEQ ID NO: 2 or 9 by deletion, substitution, or addition of 1 to 50 amino acids and that has nonsymbiotic globin activity; and
(e) a DNA comprising the nucleotide sequence shown in SEQ ID NO: 3 or 10.

13. A method for enhancing nitrogen-fixing efficiency upon plant cultivation, comprising cultivating the nodulating plant according to claim 8.
